(19) United States
(12) Patent Application Publication
(10) Pub. No.: US 2003/0216777 A1
(43) Pub. Date: Nov. 20, 2003

(54) Method of enhancing healing of interfacial gap between bone and tendon or ligament

(76) Inventors: Yin-Chun Tien, Kaohsiung (TW); Jiin-Huey Chern Lin, Winnetka, IL (US); Chien-Ping Ju, Carbondale, IL (US)

Correspondence Address: BACON & THOMAS, 625 Slaters Lane - 4th Floor, Alexandria, VA, 22314, US

(21) Appl. No.: 10/145,901

(22) Filed: May 16, 2002

(51) Int. Cl. A61B 17/08

(52) U.S. Cl. 606/214

(57) ABSTRACT

Augmentation of the tendon-bond interfacial healing, for example the tendon-bond interfacial healing in the cruciate ligament reconstruction, by filling the interfacial gap with a paste of calcium phosphate cement, a paste of bioactive glass, or a paste of calcium sulfate is disclosed.

### FIELD OF THE INVENTION

The present invention is related to augmentation of the tendon-bone interfacial healing, and in particular to augmentation of the tendon-bone interfacial healing in the cruciate ligament reconstruction.

### BACKGROUND OF THE INVENTION

In many instances of joint stability reconstructive surgery, an autografted tendon is passed through the bone tunnel. Immediately following reconstruction, biomechanical pull-out tests revealed a majority of failure points occurring at the tendon anchoring site. Therefore, the healing of the interfacial gap between bone and tendon will greatly influence the post-operative rehabilitative programs and ultimately the clinical results. Heretofore, none of surgeons or researchers has disclosed a method of enhancing a healing of an interfacial gap between a bone and a tendon by filling the interfacial gap with a paste of calcium phosphate cement a paste of bioactive glass, or a paste of calcium sulfate. The bioactive glass is also called bioglass, which is composed of calcium salts and phosphate in similar proportions as found in bone and teeth, as well as sodium slats and silicon (which are essential for bone to mineralize).

### SUMMARY OF THE INVENTION

One of the biggest disadvantages of using the hamstring tendons for reconstruction of knee cruciate ligament used to be fixation and healing. The present invention provides a potential and promising method to reinforce the fixation and to augment the tendon healing to bone in the clinical practice, which comprises filling an interfacial gap between a bone and a tendon with a paste of calcium phosphate cement, a paste of bioactive glass, or a paste of calcium sulfate. It is believed that the method of the present invention is also applicable to an interfacial gap between a bone and a ligament.

### DETAILED DESCRIPTION OF THE PREFERRED EMBODIMENTS

The preferred embodiments of the invention of the present application are:

1. A method of enhancing a healing of an interfacial gap between a bone and a tendon or ligament comprising filling the interfacial gap with a paste of calcium phosphate cement, a paste of bioactive glass, or a paste of calcium sulfate.

2. The method according to item 1 further comprising drilling a hole in said bone, and placing a portion of said tendon in said hole so that said interfacial gap is formed.

3. The method according to item 2, wherein said hole is a through hole, and said tendon is received in said through hole with two ends of said tendon protruding from two ends of said through hole.

4. The method according to item 2 further comprising drilling another hole in another bone, placing another portion of said tendon in said another hole so that another interfacial gap is formed between said another bone and said tendon, and filling said another interfacial gap with said paste of calcium phosphate cement, said paste of bioactive glass, or said paste of calcium sulfate.

5. The method according to item 4, wherein said hole and said another hole are both a through hole, and said bone and said another bone are connected with said tendon by passing said tendon through said hole and said another hole with one end of said tendon protruding from said hole and another end of said tendon protruding from said another hole.

6. The method according to item 1, wherein said interfacial gap is filled with said paste of calcium phosphate cement.

7. The method according to item 4, wherein said interfacial gap is filled with said paste of calcium phosphate cement.

8. The method according to item 7, wherein said another interfacial gap is filled with said paste of calcium phosphate cement.

9. The method according to item 6, wherein said paste of calcium phosphate cement is prepared by mixing an aqueous solution and a calcium phosphate cement comprising particles selected from the group consisting of tetracalcium phosphate, alpha-tricalcium phosphate, beta-tricalcium phosphate, calcium dihydrogen phosphate, calcium dihydrogen phosphate hydrate, acid calcium pyrophosphate, anhydrous calcium hydrogen phosphate, calcium hydrogen phosphate hydrate, calcium pyrophosphate, calcium triphosphate, calcium polyphosphate, calcium metaphosphate, anhydrous tricalcium phosphate, tricalcium phosphate hydrate, apatite, hydroxyapatite, fluorapatite, and a mixture thereof.

10. The method according to item 9, wherein said particles have a diameter of 0.05 to 100 microns, and said particles have whiskers or fine crystals on their surfaces having a width ranging from 1 to 100 nm and a length ranging from 1 to 1000 nm.

11. The method according to item 9, wherein said particles have a molar ratio of calcium to phosphate ranging from 0.5 to 2.5.

12. The method according to item 9, wherein said aqueous solution is an aqueous solution of phosphoric acid, nitric acid, hydrochloric acid, acetic acid, lactic acid, citric acid, malic acid, malonic acid, succinic acid, glutaric acid, tartaric acid, polyacrylic acid, or a salt thereof.

13. The method according to item 12, wherein said aqueous solution is an he aqueous solution of phosphate.

14. The method according to item 9, wherein per ml of said aqueous solution is mixed with 2-6 g of said calcium phosphate cement.

15. The method according to item 9, wherein said calcium phosphate cement further comprises a growth factor, a bone morphology protein or a pharmaceutical carrier.

16. The method according to item 12, wherein said aqueous solution further comprises a growth factor, a bone morphology protein or a pharmaceutical carrier.

17. The method according to item 1, wherein said filling comprises injecting said paste into said interfacial gap.

Experiment

Healing to the bone is the weakness of the hamstring graft for cruciate ligament reconstruction, most surgeons recommend a more cautions and less aggressive physical therapy program for up to 8 weeks. Calcium phosphate cement was used to augment the interface healing and the efficiency was evaluated in one of the preferred embodiments of the present invention.

Materials and Methods

Adult male New Zealand White rabbits were used for this in-vivo study. The body weights of all the rabbits were approximately 4.0 kg, and they were kept in cages measuring 90 cm×45 cm×45 cm. Activity was allowed only in the cage.

The semitendinosus tendon was chosen for anterior cruciate ligament reconstruction because of its uniformity of size and length. After the animal was anesthetized with intravenous Nembutol (45 mg/kg), the knees were approached anteromedially through the medial retinaculum. After opening the joint, the anterior cruciate ligament (ACL) was excised first. Then a bone tunnel with a diameter of 2.4 mm was made in the proximal tibia with the intrusion just anterior to medial collateral ligament (MCL) and the protrusion just at the original ACL insertion point on the tibial intercondylar spine. The femoral bone tunnel with the same size was made with the intrusion on the midline of intercondylar notch and protrusion just superior to lateral collateral ligament (LCL) origin. The semitendinosus tendon was dissected and passed through the tibial bone tunnel and femoral bone tunnel and then was sutured as a post fixation to LCL. The procedure was then done on the contra-lateral knee. Randomly, on one side of the knee, the interface between the grafted tendon and bone tunnel was filled with calcium phosphate cement. The CPC was injected into the interface by a 5 ml syringe.

The limbs were not immobilized and the rabbits were allowed activity in the cage only. In total, 22 rabbits were used for this study. The results of the study with a similar rabbit model done by Grana, et al. indicated that by 3 weeks, failure of the bone-tendon-bone in the mechanical test happened through the intraarticular portion of the graft, not as a result of pullout from the bone tunnel [Grana, W. A.; Egle, D. M.; Mahnken, R.; and Goodhart, C. W.: An analysis of autograft fixation after anterior cruciate ligament reconstruction in a rabbit model. Am J Sports Med., 3: 344-351, 1994]. Therefore, to make sure the failure point will happen at the interface between grafted tendon and bone, 6 rabbits were sacrificed respectively at the end of first and second post-operative week by overdose of Nembutal. For serial histological observation of the interface healing, 2 rabbits were sacrificed sequentially at the end of 1^{st}, 3^{rd}, 6^{th}, 12^{th} & 24^{th} post-operative week.

Calcium Phosphate Cement (CPC)

The calcium phosphate cement (CPC) used in this embodiment was obtained from mixing equimolar tetracalcium phosphate Ca₄(PO₄)₂ (TTCP) and dicalcium phosphate anhydrous CaHO₄. 2H₂O (DCPA) powders in a 25 mM phosphate-containing solution with a powder/liquid ratio of 4.0 gm/ml. The TTCP powder was fabricated in-house from the reaction of dicalcium pyrophosphate (Ca₂P₂O₇) (Sigma Chem. Co., St Louis, Mo., USA) and calcium carbonate (CaCO₃) (Katayama Chem. Co., Tokyo, Japan), while DCPA powder was a commercially available product (Janssen Chemica, Beerse, Belgium). Through a special pre-treatment process on the mixture of the original TTCP and DCPA powders prior to mixing with the phosphate-containing solution, whiskers or fine crystals were formed on the surfaces of the particles, so that the pre-treated mixture of the TTCP and DCPA powders had a capability to set in situ in an aqueous environment with sufficient strength to prevent dispersion. Details of the preparation of the CPC having whiskers or fine crystals on the surfaces of the CPC particles can be found in EP1172076 A (January 2002), which is incorporated herein by reference. The cement can be easily shaped in paste form during operation or injected into a cavity with a syringe without requiring an open way through the tissues.

Mechanical Testing of the Interfacial Helaing Tissue

The Instron material testing machine model #1322 with a 50 kg load cell was used to detect the maximal tensile strength of the healing tissue. The test samples were dissected to be a bone-tendon-bone model, the bone of which was mounted on each end. The mounting clips were designed to be tubular in shape. After inserting the femur or tibia into the tube, the bone was fixed with cross pins first and then the tube was filled with resin for fixation. While attaching the mounting clips to Instron machine, the femur was fixed at flexion of 45 degrees to align the bone tunnel along the direction of the testing force. Before starting the loading, the suture stitch which fixed the grafted tendon to LCL was removed in order to allow the interface healing tissue to be the only material to tolerate the pull strength. The original attachment of the grafted semitendinosus tendon on the tibia remained. Under this condition, the failure point was expected on the femoral bone tunnel site. The tensile strength was detected at a rate of displacement of 5.0 mm per second, until the tendon was pulled out of the femoral bone tunnel. The maximal tensile strength was collected and the data was statically analyzed by pair-t test.

Histological Study of the Interfacial Healing Tissue

The dissected knee joints were fixed in the neutralized Formalin for 72 hours first, and then decalcified with mixed solution containing 20% sodium citrate and 50% formic acid for 1 week. After decalcification, the samples were embedded in paraffin for slice cut perpendicular to the bone tunnels axis. Hematoxylin and eosin stains were done for light microscopic study.

Results

All the animals tolerated the reconstructive surgeries well. After the operation, no wound infection happened and no body weight loss observed on these rabbits.

Histological and Gross Findings

One-week specimens: In CPC group, the gross pictures of transverse sections made perpendicular to the long axis of the bone tunnel revealed the tendon-bone interface was filled out by calcium phosphate cement (CPC). Actually, the injected CPC also penetrated into the surrounding bone marrow spaces surrounding the bone tunnel. The histological pictures of these transverse sections revealed the most part of the tendon-bone interface was filled by CPC. However, thin fibrous layers observed in the seams of CPC fragments and adjacent to both the grafted tendon surface and bone tunnel surface. In non-CPC group, the gross pictures reveal the tendon-bone interface was just filled by loose tissue. These connective tissues while tested by a needle appeared to be soft, loose and fragile. In histological examination revealed these loose tissue in the interface mainly comprised of a loose fibrous tissue.

Three-week specimen: IN CPC group, the gross picture of transverse sections showed that some portions of CPC had been absorbed, and new bone ingrowth into the bone tunnel was found. The tendon-bone interface was completely filled by new growing tissues. These tissues while tested by a needle appeared to be firm with a tough consistency. Due to the new growing tissues that were so closely attached with the bone and grafted tendon that it was difficult to recognize their borders. In histological examination, many new growing bone islands were found within the CPC. Some of these new bone islands formed just on the surface of initial bone tunnel and grafted tendon and established a new continuity to them. In the non-CPC group, the gross picture still revealed the margins of bone tunnel and grafted tendon clearly. Even though the healing tissue in the interface was much denser in appearance, it could still easily torn by a needle. In histological examinations, the interface tissue showed increased production of extra-cellular matrix. Most of the collagen fibers distributed in circular orientation and surrounded the grafted tendon. However, the collagen fibers adjacent to the grafted tendon showed with irregular orientation and were woven with the fibers of the grafted tendon. And, a new continuity to the bone tunnel was only occasional observed.

Six-week specimen: In CPC group, the gross picture showed that most parts of the implanted CPC had been absorbed. It's almost impossible to locate the margins of bone tunnel and grafted tendon. The interface was totally obliterated. In histological examination, the interface was almost filled by new growing bone, and the fibers of grafted tendon were found apparently anchored onto the new growing bone. In non-CPC group, the gross picture still showed the grafted tendon-bone interface clearly, and the bone tunnel size still remained with initial size. In histological examination, some portions of collagen fibers in the interface appeared maturated and organized. However, many cysts were noticed within these fibers.

Twelve-week specimen: In CPC group, the gross pictures revealed the bone tunnels were almost healed by new growing bone and only very small portion of CPC remained. In the histological examination, most portions of the interface were filled out by new formed bone and adjacent to the surface of grafted tendon, tidemarks were observed between the grafted tendon and new formed bone. The continuity between collagen fibers of the grafted tendon and surrounding bone could be clearly observed. In non-CPC group, the interface and bone tunnel still could be clearly identified, and the bone tunnel size did not decreased much. In histological examination, no new bone formation observed and even the native bone trabeculae became thinner and looser. Massive adipose cells accumulated in the interface and bone marrow space. These findings suggested a disuse osteoporotic change.

Twenty four-week specimens: In CPC group, we could not located the original bone tunnel in most sections grossly, and only could identified the bone tunnel in some sections by the small portion of CPC which remained not to be absorbed. In histological examination, the interface was healed completely by newly ingrown bone and the continuity between collagen fibers of grafted tendon and the surrounding new grown bone had remolded and resembled Sharpey fibers. In non-CPC group, the bone tunnel still could be identified clearly. However, the bone tunnel size decreased a little bit and appeared with an irregular surface. In histological examinations, a layer of new lamellar bone formation was observed on the rim of native bone so that the bone tunnel was narrowed a little bit and appeared with irregular surface.

Mechanical Test

One-week specimen: Six specimens in the CPC group and non-CPC group respectively were tested. All the specimens failed by pullout of the tendon from the femoral bone tunnel. The mean maximal tensile strength of CPC group was 0.664±0.174 kg and the mean maximal tensile strength of non-CPC group was 0.209±0.097 kg (Table I). When tested by pair-t test, the CPC group was significantly stronger than the non-CPC group. (T=5.603, P<0.01)

Two-week specimen: Six specimens in the CPC group and non-CPC group respectively were tested. All the specimens also failed by pullout of the grafted tendon from the femoral bone tunnel. The mean maximal tensile strength of CPC group was 1.173±0.292 kg and the maximal tensile strength of non-CPC group was 0.556±0.404 kg (Table II). When tested by pair-t test, the CPC group was significantly stronger than the non-CPC group (T=3.023 P<0.05).

TABLE I
Maximal Tensile Strength (1 week)

| | With Cement | Without Cement |
|---|---|---|
| 1 | 0.673 (kg) | 0.133 (kg) |
| 2 | 0.464 | 0.0165 |
| 3 | 0.610 | 0.321 |
| 4 | 0.511 | 0.212 |
| 5 | 0.915 | 0.096 |
| 6 | 0.810 | 0.327 |
| Mean | 0.6638 ± 0.1736 | 0.2090 ± 0.0969 |

T = 5.603
P < 0.01

TABLE II
Maximal Tensile Strength (2 week)

| | With Cement | Without Cement |
|---|---|---|
| 1 | 1.358 (kg) | 0.874 (kg) |
| 2 | 1.304 | 0.473 |
| 3 | 1.406 | 0.103 |
| 4 | 0.664 | 0.119 |
| 5 | 0.976 | 0.667 |
| 6 | 1.327 | 1.102 |
| Mean | 1.1725 ± 0.2923 | 0.5563 ± 0.4036 |

T = 3.028
P = 0.013 < 0.05

From the histological results, two prominent differences between CPC group and non-CPC group were observed.

The first difference was the way by which the bone ingrowth in the tendon-bone interface was formed. In the non-CPC group, usually the new bone formation was observed only on the margin of bone tunnel and the new bone grew slowly with lamellar appearance. The Sharpey's fiber continuity was established by the advancing bone formation onto the maturated collagen fiber in the tendon-bone interface. In the CPC group, through the supreme osteoconductive character of this synthetic bone graft, the new bone formation appeared much earlier, rapid and extensive. The new grown bone islands with woven bone appearance were distributed all over the tendon-bone interface and later they filled out the interface.

The second difference was the location where the Sharpey's fibers were established. In non-CPC group, the layer of new bone formation usually was limited with only a thin layer of lamellar bone lining on the bone tunnel. So, the Sharpey's fibers happened just adjacent to the bone tunnel and this was the reason why the bone tunnel size while observed grossly remained with the same size even in the late stage. In CPC group, the interface usually was filled out by new formed bone and the Sharpey's fiber was observed just adjacent to the grafted tendon surface and this was the reason why the bone tunnel size while observed grossly appeared to be so small.

Although the present invention has been described with reference to specific details of certain embodiments thereof, it is not intended that such details should be regarded as limitations upon the scope of the invention except as and to the extent that they are included in the accompanying claims. Many modifications and variations are possible in light of the above disclosure.

What is claimed is:

1. A method of enhancing a healing of an interfacial gap between a bone and a tendon or ligament comprising filling the interfacial gap with a paste of calcium phosphate cement, a paste of bioactive glass, or a paste of calcium sulfate.

2. The method according to claim 1 further comprising drilling a hole in said bone, and placing a portion of said tendon in said hole so that said interfacial gap is formed.

3. The method according to claim 2, wherein said hole is a through hole, and said tendon is received in said through hole with two ends of said tendon protruding from two ends of said through hole.

4. The method according to claim 2 further comprising drilling another hole in another bone, placing another portion of said tendon in said another hole so that another interfacial gap is formed between said another bone and said tendon, and filling said another interfacial gap with said paste of calcium phosphate cement, said paste of bioactive glass, or said paste of calcium sulfate.

5. The method according to claim 4, wherein said hole and said another hole are both a through hole, and said bone and said another bone are connected with said tendon by passing said tendon through said hole and said another hole with one end of said tendon protruding from said hole and another end of said tendon protruding from said another hole.

6. The method according to claim 1, wherein said interfacial gap is filled with said paste of calcium phosphate cement.

7. The method according to claim 4, wherein said interfacial gap is filled with said paste of calcium phosphate cement.

8. The method according to claim 7, wherein said another interfacial gap is filled with said paste of calcium phosphate cement.

9. The method according to claim 6, wherein said paste of calcium phosphate cement is prepared by mixing an aqueous solution and a calcium phosphate cement comprising particles selected from the group consisting of tetracalcium phosphate, alpha-tricalcium phosphate, beta-tricalcium phosphate, calcium dihydrogen phosphate, calcium dihydrogen phosphate hydrate, acid calcium pyrophosphate, anhydrous calcium hydrogen phosphate, calcium hydrogen phosphate hydrate, calcium pyrophosphate, calcium triphosphate, calcium polyphosphate, calcium metaphosphate, anhydrous tricalcium phosphate, tricalcium phosphate hydrate, apatite, hydroxyapatite, fluorapatite, and a mixture thereof.

10. The method according to claim 9, wherein said particles have a diameter of 0.05 to 100 microns, and said particles have whiskers or fine crystals on their surfaces having a width ranging from 1 to 100 nm and a length ranging from 1 to 1000 nm.

11. The method according to claim 9, wherein said particles have a molar ratio of calcium to phosphate ranging from 0.5 to 2.5.

12. The method according to claim 9, wherein said aqueous solution is an aqueous solution of phosphoric acid, nitric acid, hydrochloric acid, acetic acid, lactic acid, citric acid, malic acid, malonic acid, succinic acid, glutaric acid, tartaric acid, polyacrylic acid, or a salt thereof.

13. The method according to claim 12, wherein said aqueous solution is an aqueous solution of phosphate.

14. The method according to claim 9, wherein per ml of said aqueous solution is mixed with 2-6 g of said calcium phosphate cement.

15. The method according to claim 9, wherein said calcium phosphate cement further comprises a growth factor, a bone morphology protein or a pharmaceutical carrier.

16. The method according to claim 12, wherein said aqueous solution further comprises a growth factor, a bone morphology protein or a pharmaceutical carrier.

17. The method according to claim 1, wherein said filling comprises injecting said paste into said interfacial gap.
